(19) United States
(12) Patent Application Publication
(10) Pub. No.: US 2026/0146276 A1
(43) Pub. Date: May 28, 2026

(54) SYSTEMS AND METHODS FOR TRACKING A PROCESS

(71) Applicants: Jaren Faleide, Denver, CO (US); Ronald Faleide, Denver, CO (US)

(72) Inventors: Jaren Faleide, Denver, CO (US); Ronald Faleide, Denver, CO (US)

(21) Appl. No.: 19/397,466

(22) Filed: Nov. 21, 2025

Related U.S. Application Data
(60) Provisional application No. 63/725,308, filed on Nov. 26, 2024.

(51) Int. Cl. C12Q 1/02

(52) U.S. Cl. CPC C12Q 1/02

(57) ABSTRACT

A system for tracking stages for tracking stages of a process at multiple locations. The system comprises a mounting assembly, a sheet retaining assembly and a tracking assembly. The mounting assembly comprises a mounting bracket configured and dimensioned to attach to an intended surface of a container. A connection structure(s) is disposed on an exterior side of the mounting bracket and is configured to define a removable mating engagement with a corresponding connection structure(s) of a support plate of the sheet retaining assembly. The sheet retaining assembly is configured to retain induvial retaining sheets and may be disposed and removed from individual containers at various locations. A user may update individual tracking sheet(s) disposed onto a sheet retaining assembly with information associated with a product and may move and place that same sheet to another location associated with a different step.

This U.S. Non-Provisional Patent Application claims the benefit of priority from U.S. Provisional Patent Application Ser. No. 63/725,308, filed Nov. 26, 2024, the entire disclosure of which is hereby incorporated by reference.

### FIELD

Embodiments of the present disclosure relate to systems and methods for tracking the steps of a process at multiple locations and increments, including using a removable sheet retaining assembly with a tracking sheet(s) and temporarily placing it on multiple intended locations associated with different steps of the process.

### BACKGROUND

Various types of processes often require that an individual(s) physically go to and from various locations to complete tasks, such as taking notes pertaining to observed information, and/or to complete checklists associated with several steps of a process, including to ensure quality. Such processes may include, without limitation, wine making, brewing processes, liquor distillation processes, or in general, processes for creating a product(s) in a laboratory. As it relates to wine making, raw fruit and/or a resulting product may be processed at different locations. For example, a fruit press may be in a first area, whereas fermentation containers may be in a different location. Tasks relating to gathering information and performing checklists often require that the user(s) physically move from one location to another, or in some cases that the user(s) physically move components, e.g., equipment or the product itself, from one location to another. Such tasks can become cumbersome and can lead to human error and associated delays, including inaccuracies with notetaking at different locations after several days or weeks. The foregoing challenges are compounded by the fact that notetaking and completing checklists often requires physically gathering information, sometimes complex, on various mediums, which may be difficult to organize and retrieve, especially between different locations. In such contexts, there is a potential for misplacement of information.

Given the foregoing, there is a need to provide a tracking system that overcomes these drawbacks and provides other advantages. Various industries, including the wine making industry, would benefit by providing a tracking system that would allow a user to conveniently gather information and complete checklists associated with a process involving various steps at multiple locations over time. A benefit would be realized by providing a tracking system with mounting brackets that may be disposed at various locations, including equipment and/or containers, and onto which a removable sheet retaining assembly could be temporarily placed. An even further benefit would be realized if such a tracking system included a tracking assembly with individual tracking sheets that could be disposed on the sheet retaining assembly and that could be updated with information associated with various stages of a product. Yet a further benefit would be provided if such a tracking system would also comprise an archive sheet onto which individual product sheets would be compiled.

### SUMMARY

Embodiments of the present disclosure are directed to systems and methods for tracking stages of a process at multiple locations. According to one embodiment of the present disclosure, a tracking system generally comprises a mounting assembly, a sheet retaining assembly and a tracking assembly. The mounting assembly is generally intended to serve as a structure onto which a sheet retaining assembly may be temporarily placed. As such, in one or more embodiments the mounting assembly comprises a mounting bracket with a substantially concave configuration, at least one connection structure disposed on an exterior side of the mounting bracket, and at least one attachment structure disposed on an interior side of the mounting bracket. The attachment structure is configured to attach at least the mounting bracket to an intended surface, which may be an item in a location associated with a given step of the process, e.g., a container or other type of equipment.

In various embodiments, the sheet retaining assembly comprises a support plate with a retainer operatively disposed thereon, and a connection structure(s) disposed on a reverse side of the support plate. The connection structure(s) of the mounting assembly is operatively configured with the at least one connection structure(s) of the sheet retaining assembly to collectively define a removable mating engagement between them. In various embodiments, the tracking assembly comprises an individual tracking sheet that is removably disposable onto a front side of the support plate.

In various embodiments, each step of the process is associated with an individual tracking sheet(s) that a user may use to monitor or update information associated with any given step of the process. Such information, may include, without limitation, timeframes, ingredients, equipment required, temperature or humidity conditions, general notes, and/or the date(s) in which the step is conducted. Such information may also include, without limitation, associated checklists and/or fields for the user to input additional information manually and/or automatically, for example, via a mobile device operatively configured with an electronic platform.

Although the inventive tracking system contemplates using a physical individual tracking sheet(s) and/or physical archive sheet(s), alternative embodiments of the present disclosure contemplate using a mobile device operatively configured with an electronic platform, database and/or server for the user to automatically retrieve, monitor and/or virtually update information associated with the process. For example, each intended location associated with a step of the process may comprise a digital component, e.g., barcode or QR code, that may be scanned with a camera of the device to gather and/or update information associated with the specific step.

Various concepts disclosed herein may be provided in combination with one another even if such combination is not specifically depicted or described.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments according to the present disclosure, exemplary methods and/or materials are described below. In addition, the materials, methods, and examples are illustrative only and are not intended to be necessarily limiting.

### DESCRIPTION OF THE DRAWINGS

Those of skill in the art will recognize that the following description is merely illustrative of the principles of the disclosure, which may be applied in various ways to provide many different alternative embodiments. This description is made for illustrating the general principles of the teachings of this disclosure and is not meant to limit the inventive concepts disclosed herein.

The accompanying drawings, which are incorporated in and constitute a part of the specification, illustrate embodiments of the disclosure and together with the general description of the disclosure given above and the detailed description of the drawings given below, explain the principles of the disclosure.

FIG. 1 is a front view of one embodiment of a tracking system according to the present disclosure comprising two sheet retaining assemblies.

FIG. 2 is a front disassembled view of one embodiment of a tracking system according to the present disclosure comprising a mounting assembly, a sheet retaining assembly and a tracking assembly.

FIG. 3 is a rear view of embodiments of two mounting brackets of a mounting assembly and two support plates of a sheet retaining assembly of the tracking system according to the present disclosure.

FIG. 4 is a front perspective view of one embodiment of a mounting bracket of the mounting assembly of the tracking system according to the present disclosure.

FIG. 5 is a front view of one embodiment of a mounting bracket of the mounting assembly of the tracking system according to the present disclosure.

FIG. 6 is a top perspective view of one embodiment of a mounting assembly disposed on a surface.

FIG. 7 is a front perspective view of one embodiment of a sheet retaining assembly of the tracking system according to the present disclosure.

FIG. 8 is a rear perspective view of one embodiment of a sheet retaining assembly of the tracking system according to the present disclosure.

FIG. 9 is a diagrammatic representation of the steps of one embodiment of the method of using a tracking system according to the present disclosure.

FIG. 10 is a block diagram of a computing environment associated with embodiments of a system according to the present disclosure.

The drawings are not necessarily to scale. In certain instances, details that are not necessary for an understanding of the disclosure or that render other details difficult to perceive may have been omitted. It should be understood, of course, that the disclosure is not necessarily limited to the embodiments illustrated herein.

### DETAILED DESCRIPTION

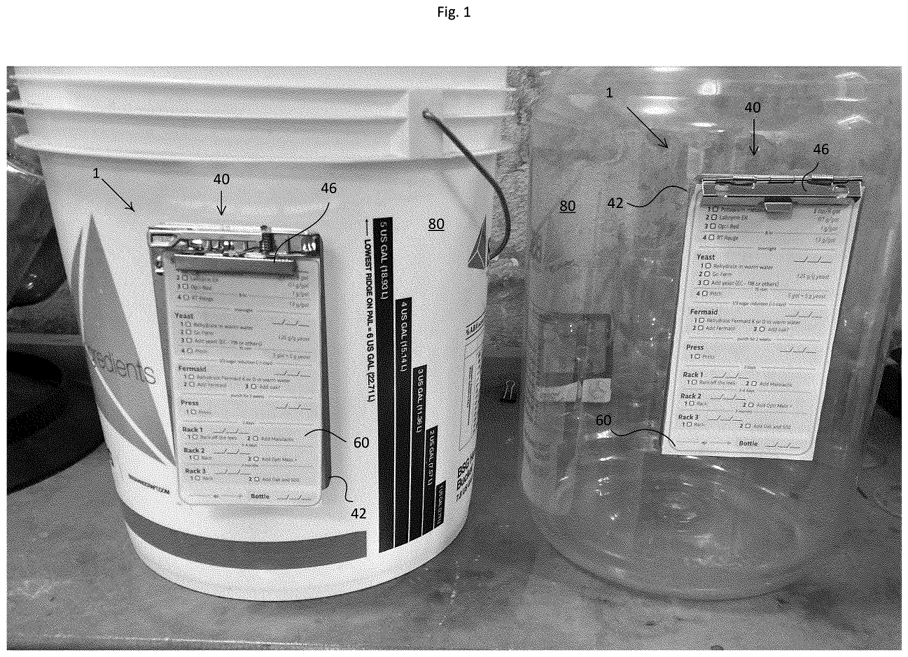

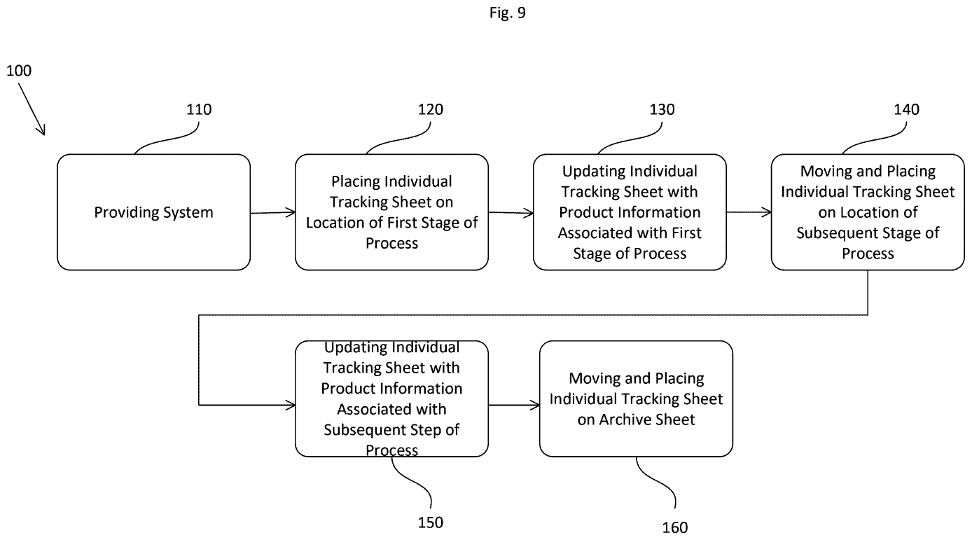

With initial reference to FIGS. 1-8 and 10, embodiments of the present disclosure are directed to a system 1 for tracking various stages of a process. With reference to FIG. 9, other embodiments of the present disclosure are also directed to a method 100 for tracking various stages of a process. The tracking system 1 according to one or more embodiments of the present disclosure may be used as a monitoring tool for a variety of processes, including without limitation, the production of wine and other liquors, including via a fermentation or distillation processes, but also the production of other products at chemistry labs, manufacturing or production facilities, or other settings where it may be necessary to track various stages of production of a product(s) at more than one location. As used herein, a “product” or “resulting product” may refer to an intermediate product or final product according to any given stage(s) of the process, including without limitation, wine, liquors, distillates, chemical products whether in liquid or solid form, etc. As such, one or more embodiments contemplate that individual containers or equipment associated with the product(s) may be moved to different locations as a part of an underlying process, for example, to control temperature, humidity, sunlight, or for convenience or proximity to ingredients and/or equipment, etc. Accordingly, in one or more embodiments the inventive system 1 incorporates removable components that a user may independently move between various locations to gather information or data characteristics associated with the process.

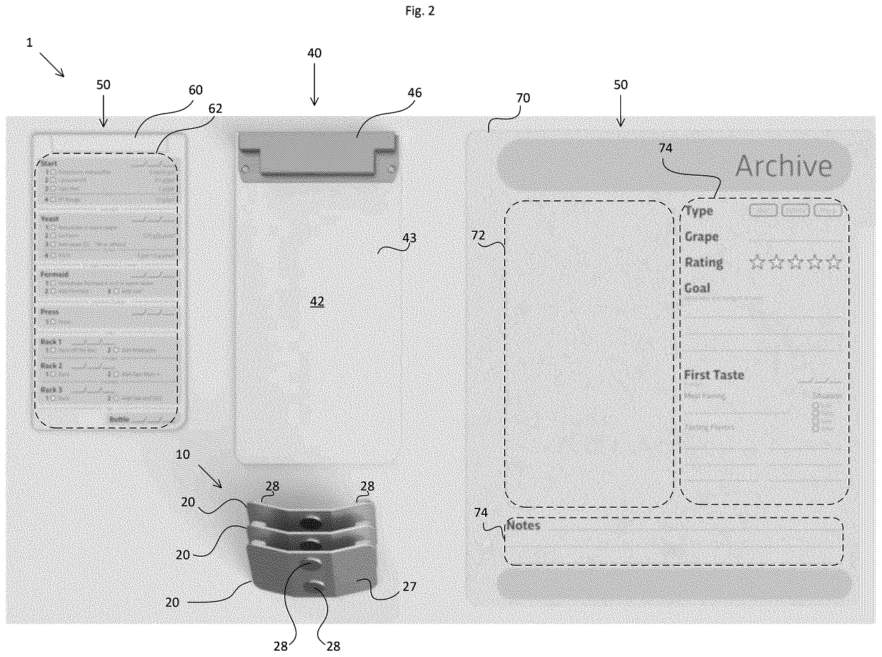

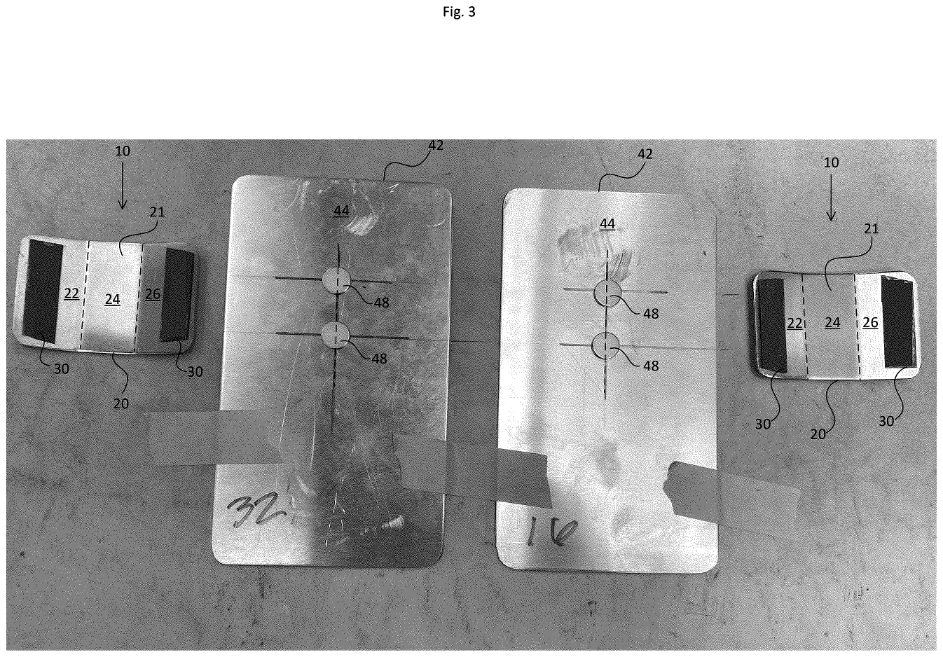

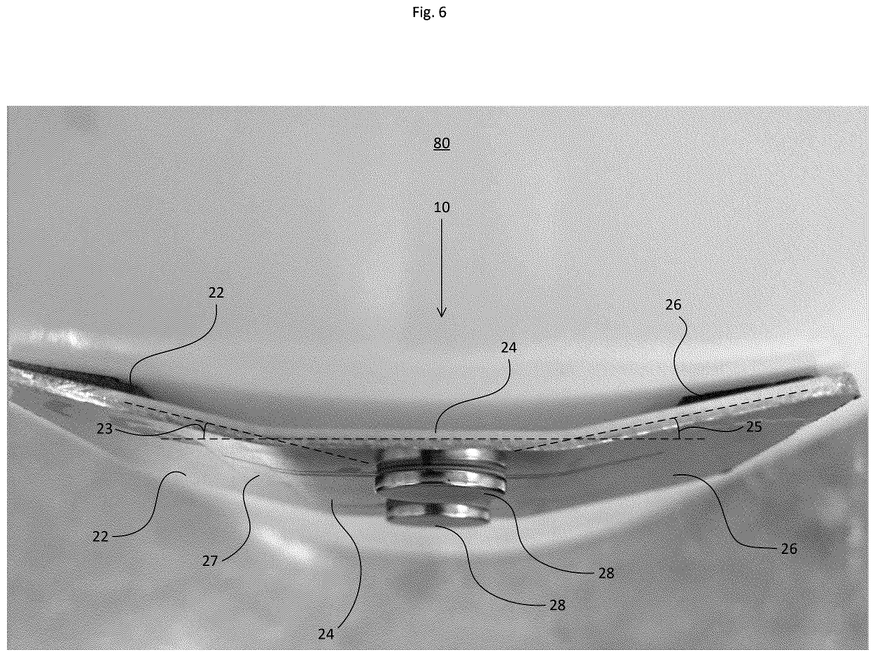

With reference to at least FIG. 2, in one or more embodiments, the tracking system 1 comprises a mounting assembly 10, a sheet retaining assembly 40 and a tracking assembly 50. The mounting assembly 10 is intended to be disposed on an intended surface 80, e.g., a container or other related structure or equipment that is associated with a predetermined stage or step of the process. The mounting assembly 10 generally comprises a mounting bracket 20 with a configuration or shape that substantially corresponds to the shape of the intended attachment surface, e.g., a container surface 80, as shown in FIGS. 1 and 4-6. In at least one embodiment according to the present disclosure, for example, as is shown at least in FIGS. 3 and 6, the mounting bracket 20 comprises a substantially concave shape that is structured and dimensioned to facilitate an attachment to an intended cylindrical container surface 80.

As is perhaps best show in FIG. 3, in one or more embodiments of the system 1 according to the present disclosure, the mounting assembly 10 comprises two attachment structures 30 disposed on the interior side 21 of the mounting bracket 20. In the illustrative embodiment of FIG. 3, the attachment structures 30 are disposed in spaced apart relation to one another with the attachment structures 30 being disposed respectively on a first segment 22 and on a third segment 26 of the mounting bracket 20. However, this is not necessarily limiting as the attachment structure(s) 30 may be disposed at other locations and/or sections of the interior side 21 of the mounting bracket 20, for example, on a second segment 24. The attachment structure(s) 30 generally comprises a mechanism that attaches the mounting bracket 20 to the intended container surface 80, including without limitation, two-sided tape, epoxy, glue, or related attachment mechanisms.

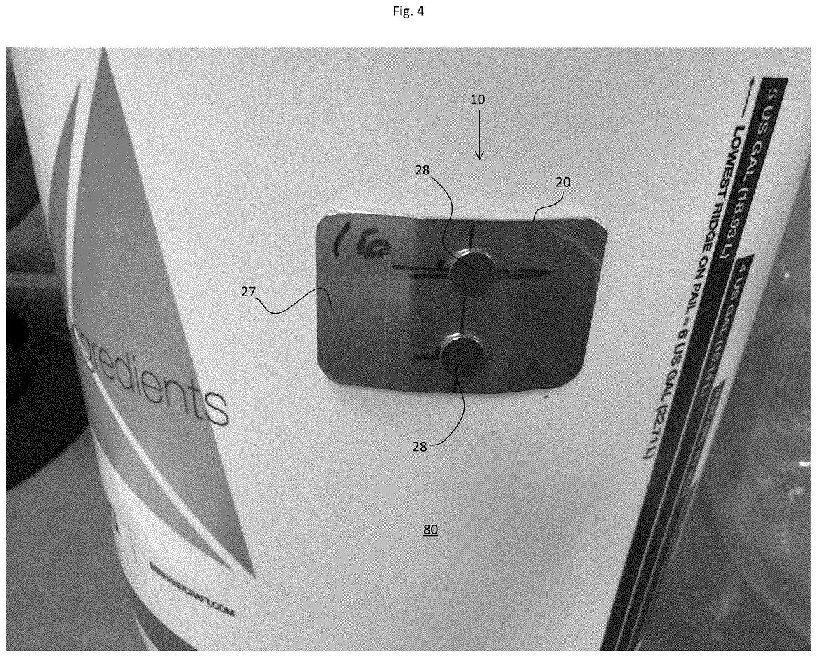

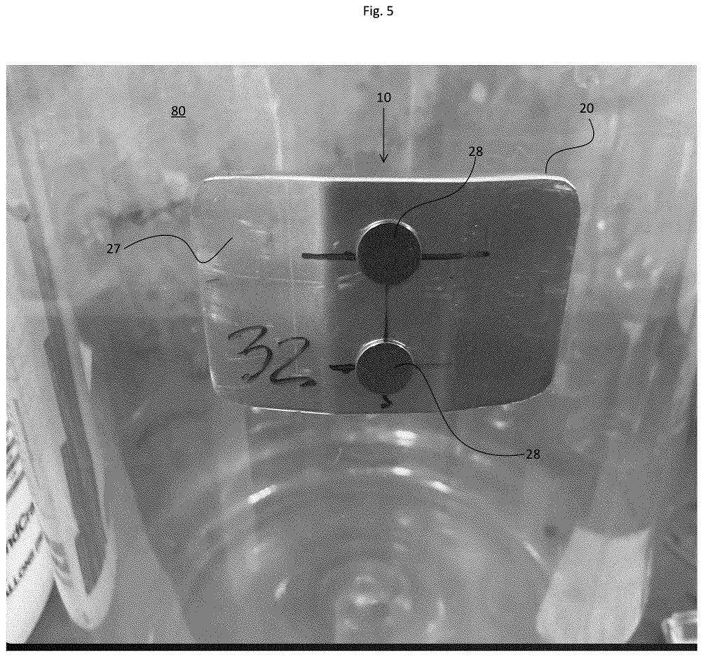

As is perhaps best shown in FIGS. 4-5, in one or more embodiments the mounting bracket 20 comprises a connection structure(s) 28, which as will be explained later, is structured to form a removable retaining engagement with connection structure(s) 48 of the support plate 42 of the retaining assembly 40. In one or more embodiments of the system 1, the connection structure(s) 28 of the mounting assembly 10 is disposed on a second or exterior side 27 of the mounting bracket 20. For example, the illustrative embodiment of FIGS. 4 and 5 show a mounting assembly 10 with two connecting structures 28 disposed on the exterior side 27 of the mounting bracket 20 around a second segment 24 of the mounting bracket 20. As shown in FIGS. 2 and 4-6, in some embodiments the connecting structures 28 are disposed in a spaced apart relation to one another and are aligned along a vertical axis and on the second segment 24. This arrangement may be beneficial, including to achieve stability of the sheet retaining assembly 40 when it is disposed on the mounting bracket 20. However, this arrangement is not necessarily limiting as other embodiments contemplate different arrangements of the connecting structures 28 on the mounting bracket 20 are also possible.

As briefly mentioned above and as is perhaps best shown in the illustrative embodiment of FIG. 6, which shows a mounting bracket 20 disposed on an intended surface, the connecting structures 28 are generally disposed on an exterior side 27 of the mounting bracket 20. However, in an alternative embodiment, the connection structure(s) 28 are attached directly to the intended surface. As contemplated by one or more embodiments of the system 1, the mounting bracket 20 comprises a plurality of segments, e.g., 22, 24 and 26, that collectively define the substantially curved or concave shape thereof. For example, and as is perhaps best shown in FIGS. 3 and 6, in one or more embodiments of the system 1 the mounting bracket 20 comprises a first segment 22 connected to a second segment 24, and a third segment 26 connected to the second segment 24.

As shown in FIGS. 2-6, in various embodiments the first segment 22, the second segment 24 and the third segment 26 collectively define a substantially concave shape of the mounting bracket 20. As is perhaps best shown in FIG. 6, in at least one embodiment the segments 22 and 26 are disposed at an angle 25 with respect to the plane of the second or central segment 24. In one or more embodiments, the angle between the first segment 22 and the central segment 24 is substantially equivalent to the angle between the third segment 26 and the central segment 24. Also, in the illustrative embodiments of FIGS. 2-6 the second segment 24 is disposed in a spaced apart relation to the intended container surface 80, with the first segment 22 and the third segment 26 being interconnected to the intended surface via a corresponding attachment structure 30.

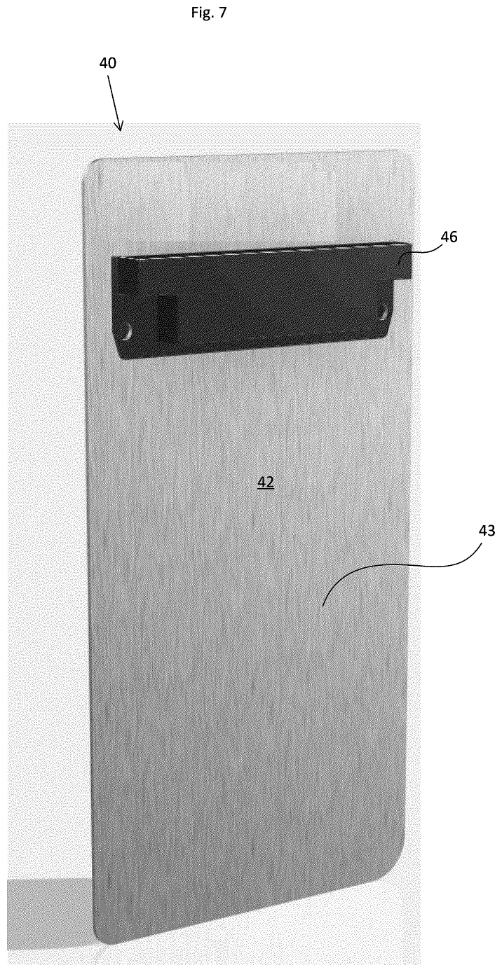

As briefly mentioned above, in various embodiments the tracking system 1 according to the present disclosure comprises a sheet retaining assembly 40. As is perhaps best shown in the illustrative embodiments of FIGS. 2 and 7, the sheet retaining assembly 40 generally comprises a support plate 42 with a retainer 46 operatively disposed on a front side 43 thereon. In one or more embodiments, the retainer 46 is configured to hold an individual tracking sheet(s) 60 on the front side 43. The support plate 42 is generally configured and dimensioned such that the geometry and/or size of the front side 43 is at least equivalent to that of the individual tracking sheet(s) 60. As such an individual tracking sheet(s) 60 may be retained on the sheet retaining assembly 40 and either or both of the tracking sheet(s) 60 and the retaining assembly 40 may be moved together between intended locations. While it is contemplated that the sheet retaining assembly 40 be operable retain at least one individual tracking sheet 60, in at least one embodiment it is operable to retain multiple individual tracking sheets 60, for example, if a user needs to ascertain data and/or perform a checklist at multiple intended locations, and including with individual tracking sheets 60 being dedicated to each intended location.

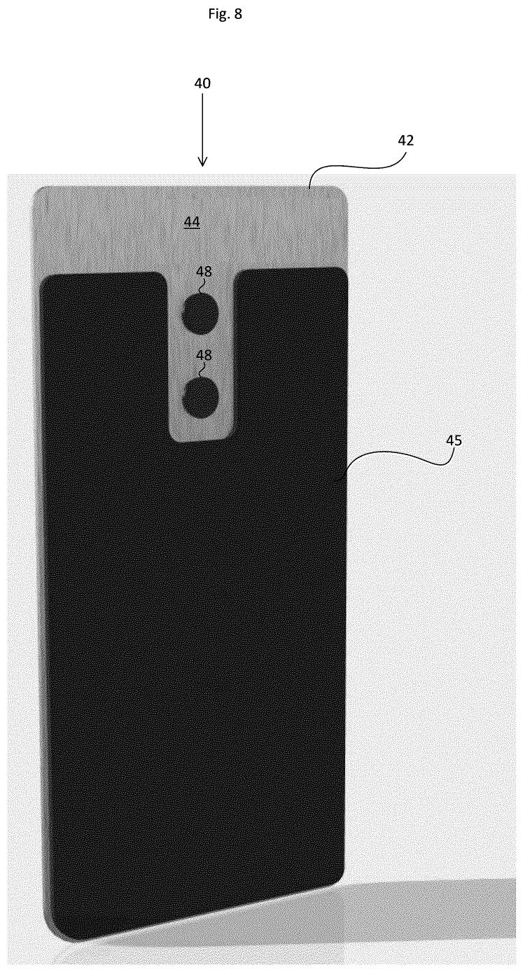

With reference now to FIGS. 3 and 8, the support plate 42 of the sheet retaining assembly 40 generally comprises a reverse side 44 for removable attachment to a mounting bracket(s) 20. The reverse side 44 of the support plate 42 is the side that is disposable onto the mounting bracket 20 of an individual item, e.g., a container or other equipment, including during a specific step of the process, for example, to gather data and/or complete a checklist during that step. Thus, in one or more embodiments, the support plate 42 may be initially disposed onto a container or equipment and may be moved later to a different container(s). By way of example, in at least one embodiment the connecting structures 28 and/or 48 comprise a removable attachment mechanism, including without limitation, clips and/or brackets, a hook and loop attachment mechanism, magnets, etc. As shown in the illustrative embodiments of FIGS. 3 and 8, the connecting structure(s) 48 of the sheet retaining assembly 40 is generally disposed on the reverse side 44 of the support plate 42. As is also shown in FIGS. 3 and 8, in some embodiments the connecting structures 48 are disposed in a spaced apart relation to one another and aligned along a vertical axis and their location and/or separation substantially corresponds to the separation of the connecting structures 28 of the mounting bracket 20.

As is further shown in FIGS. 3 and 8, in one or more embodiments the connecting structure(s) 48 are disposed around a top section of the reverse side 44 of the support plate 42, including for stability. However, this is not necessarily limiting as the connecting structures 48 may be disposed elsewhere on the reverse side 44 of the support plate 42. In addition, a vertical alignment of the connecting structures 28, e.g., as shown in FIGS. 2 and 4-6, and/or the connecting structures 48, e.g., as shown in FIGS. 3 and 8, is not strictly required as various embodiments according to the present disclosure also contemplates a horizontal alignment or a combination of a horizontal and vertical alignments. In various embodiments, the connecting structures 48 and corresponding connecting structures 28 are disposed in a substantially similar alignment, including to at least partially enable a stable connection between them. As such, and as is shown in the illustrative embodiment of FIG. 1, individual sheet retaining assemblies 40 may be removably disposed on intended containers 80.

As is further shown in the illustrative embodiment of FIG. 8, in some embodiments a pad 45 is disposed the reverse side 44 of the support plate 42, for example to provide a predetermined offset such that the support plate 42 is upright and does not move when disposed on a container. In embodiments, the pad 45 is also structured to protect the container and/or support plate 42. By way of example, in some embodiments, the pad 45 comprises a soft material, such as foam, rubber and/or cork. In some embodiments, the pad 45 is dimensioned to account for the geometry of the reverse side 44 of the support plate 42 and/or the location of the connecting structures 48. By way of example, as shown in the illustrative embodiment of FIG. 8, in embodiments the pad 45 has a cut-out at the approximate location of the connecting structures 48.

With reference to FIGS. 1 and 2, and as briefly mentioned above, the tracking system 1 according to one or more embodiments of the present disclosure comprises a tracking assembly 50. The tracking assembly 50 generally comprises an individual tracking sheet 60 and/or an archive sheet 70. As mentioned above, the individual tracking sheet 50 is intended to be removably placed on the sheet retaining assembly 40, either before or after the user places the sheet retaining assembly 40 on a mounting bracket(s) 20, e.g., to gather data and/or perform a checklist associated with a step of the process. As briefly mentioned above, in one or more embodiments the individual tracking sheets 60 comprise a section 62 with fields for a user complete with product information and/or for the user to review and/or conduct a checklist. As it relates to winemaking, such fields may relate to information about various stages or steps of the wine production process, for example, what ingredients are added at the first step or start of the process, during a second step or yeast stage, during a third step or fermentation step, during a fourth step and/or a press stage, or before bottling.

For example, and with reference to the illustrative embodiments of FIGS. 1 and 2, the start or initial stage comprises a checklist and/or information relating to various quantities of one or more of sulfites, e.g., potassium meta sulfite, enzyme preparations to control color intensity, e.g., Lallyzyme EX, a yeast derivative nutrient, e.g., Opti-red, or RT Rouge. In at least one other embodiment, the start or initial stage also comprises a field for inputting a specific date in which it was conducted or wait time indications, for example, a wait time of about 8 hours between adding Opti-red and RT Rouge. As a further example, in one embodiment a second or yeast stage comprises a checklist and/or information relating to methods of application of the yeast, e.g., rehydrate in warm water, etc., adding nutrients to the yeast, e.g., Go-Ferm® yeast rehydration nutrient, adding the yeast, e.g., EC—118 or other, or about the pitch ration, e.g., about 5 grams of yeast to about 5 gallons of liquid. In the embodiments shown in FIGS. 1-2, the yeast stage also comprises a field for inputting the specific date of the step or other information, for example, associated with wait times, e.g., about a 1/3 sugar reduction within 3 days.

In one or more embodiments, the third stage or fermentation stage comprises a checklist and/or information relating to the fermentation stage, e.g., rehydrating and/or adding various fermentation nutrients such as FERMAID™ K or FERMAID™ O in warm water or adding other ingredients such as floating oak. In at least one embodiment, the third stage comprises information relating to the date(s) in which the fermentation was conducted or checked off, and/or the duration of the fermentation process, e.g., about two weeks. In an embodiment, a fourth stage or press stage comprises a checklist and/or information relating to pressing the wine, for example in a fruit or other press. This step, however, may be conducted out of sequence, for example, before, after and/or during either of the preceding steps.

A fifth stage or racking stage generally comprises information and/or a checklist associated with transferring the wine from its fermentation container to another container. This step may also be referred to as racking the wine off the lees, or separating the wine from particles associated with the fermentation process, i.e., yeast residue, foam, proteins, oak particles, buildup, etc. Accordingly, in at least one embodiment this step also comprises information and/or a checklist relating to the extraction date and/or adding other ingredients, such as malolactic bacteria, OPTI'MALO PLUS™, oak and/or sulfur dioxide (SO₂). In an embodiment, the racking stage information and/or checklist also relate to the date(s) in which any of these other ingredients were added. In at least another embodiment, a last field comprises information associated with the date in which the wine was bottled and/or its age.

With reference again to FIG. 2, and as briefly mentioned above, in at least one embodiment according to the present disclosure, the tracking system 1 comprises an archive sheet 70. As shown in FIG. 2, in at least one embodiment, the archive sheet 70 comprises a placeholder 72 for a section 62 of the tracking assembly 60 to be placed. By way of example, in some embodiments the tracking assembly 60 comprises a sticker or other removable element that is disposable on the placeholder 72. In at least one embodiment, the archive sheet 70 comprises a section for inputting additional product information 74. As it relates to winemaking, such additional product information 74 and/or fields of input generally relate to the type of wine, e.g., red, white, rosé, the type of grape used, e.g., malbec, merlot, cabernet, pinot noir, muscat, etc., the rating, e.g., 1 to 5 stars, or goal associated with the specific wine. In at least one embodiment, for example as is shown in FIG. 2, as it relates to winemaking, the product information 74 comprises information and/or fields relating to characteristics associated with a first taste of the wine, or other information including, without limitation, meal pairing, tasting flavors, or a specific situation or context, e.g., a solo tasting, a party, a date or other. The archive sheet 70 may optionally comprise a field 74 for the user to input additional notes. As such, multiple archive sheets 70 associated with different products may be compiled and kept in a single location. Such additional notes may include information about the final product, which may be in addition to information about the production of the wine.

With reference now to FIG. 9 and as mentioned above, the present disclosure is also directed to a method 100 of using the system 1 as described herein to track various stages of a process. As shown at 110, the method 100 according to the present disclosure comprises providing as system 1 as described herein. As shown at 120, the method 100 comprises placing an individual tracking sheet 60 on an intended location of the first stage of the process. As shown at 130, the method 100 further comprises updating the individual tracking sheet 60 with product information 62 associated with the first stage of the process. Thereafter, as shown at 140, the method 100 further comprises moving and placing the individual tracking sheet 60 at a location associated with a subsequent stage of the process. As shown at 150, the method 100 further comprises updating the individual tracking sheet 60 with product information 62 associated with a subsequent step of the process. Steps 140 and/or 150 may be repeated on multiple locations associated with subsequent stages of the product until the process is complete or at least partially complete. As shown at 160 in FIG. 9, in some embodiments the method 100 optionally comprises moving and placing an individual tracking sheet on an archive sheet 70.

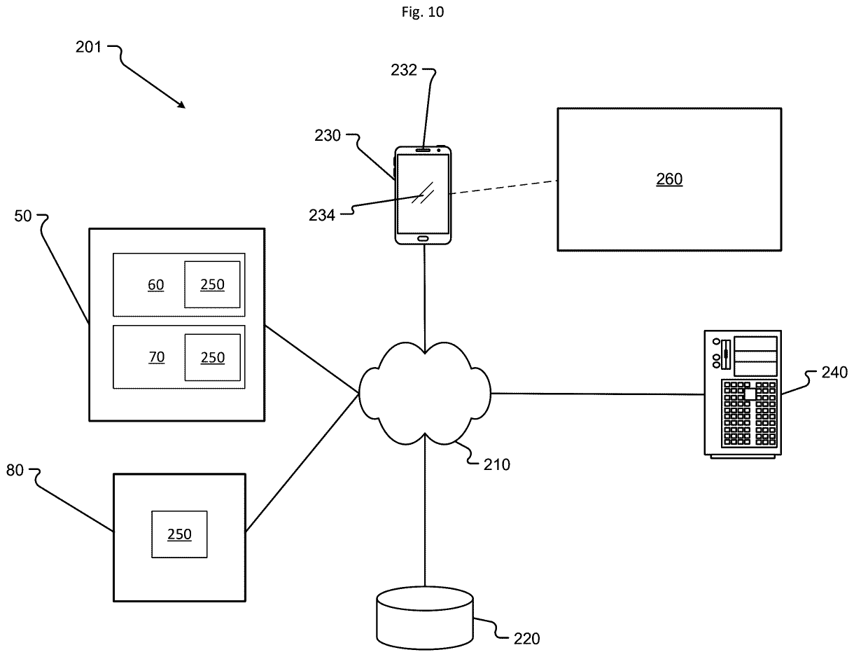

With reference to FIG. 10, further embodiments according to the present disclosure contemplate providing a tracking system 201 comprising an electronic platform 260 to monitor the steps of a process. Some embodiments of the system 201 contemplate incorporating a tracking assembly 50 comprising a digital component 250 disposed on the container surface 80, with the digital component 250 being operatively configured with an electronic platform 260 for a user to gather, monitor, input or edit product information 62 and/or 72 as described above. For example, in at least one embodiment, the tracking system 201 comprises an electronic platform 260 that may be accessed via a mobile or desktop device 230. In one or more embodiments, the mobile device 230 comprises any one of a mobile telephone, smartphone, tablet, laptop computer, smartwatch, etc. In some embodiments, the electronic platform 260 comprises a downloadable mobile application and/or executable code operatively configured with a browser to allow a user to access an electronic module to virtually track, input and/or update product information 62 and/or 72. Such product information 62 and/or 72 may be saved and/or stored on a server 240 and/or an electronic database 220 through a cloud or network 210 and may be accessible in real time via a mobile device 230, for example by scanning a digital component 250 disposed on individual containers 80. For example, in some embodiments at least some containers associated with the specific production process are provided with a unique digital component 250 such as a QR code, barcode, numeric code, etc., that may be scanned or ascertained with the camera 232 of a mobile device 230.

As shown in FIG. 10, in one or more embodiments according to the present disclosure, a digital component 250 is printed on an individual tracking sheet(s) 60 and/or an archive sheet(s) 70. In one embodiment, upon a successful scan of the digital component 250, the user can access the electronic platform 260 via the device 230 to track, input and/or update product information 62 and/or 72 without needing to manually write the information on a physical tracking sheet(s) 60 and/or an archive sheet(s) 70. For example, the electronic platform 260 may visually reproduce a window, e.g., on the screen 234 of a device 230. In one or more embodiments, the window comprises information, checklists, fields, etc., including substantially as described above. In at least one embodiment, data associated with any given step is communicated in real time to the database 220 and/or server 240, and may be accessed from another location, for example, via the same or another mobile device 230 and/or desktop device, including for further updating, editing, exporting, etc. As such, a user may physically move to the location of a first intended stage to track, input and/or update product information 62 and/or 72 associated with that first intended stage. The user may later move to the location of another, subsequent stage(s) to track, input and/or update product information 62 and/or 72 associated with the subsequent stage(s). In one embodiment, each stage is associated with a unique window and/or tab that corresponds to a step that can be automatically accessed through the electronic platform by scanning a digital component 250. Alternatively, the user may directly select and/or access any given window or tab associated with a step, e.g., from a predetermined list or a user created list, or by manually inputting a unique identifier associated with the intended step(s), such as a numerical code, a letter(s) or combinations thereof. As such, the user can perform a manual input and/or update to the fields and/or information 62 and/or 72 within the electronic platform 260 without needing to scan a digital component.

In an even further embodiment according to the present disclosure, the user may use the mobile device 230 to update the electronic database 220 with information 62 and/or 72 already associated with an individual tracking sheet(s) 60 and/or an archive sheet(s) 70. For example, a user may use the camera 234 of the device to scan product information 62 and/or 72 already present respectively in an individual tracking sheet(s) 60 and/or an archive sheet(s) 70. In an embodiment, such information is automatically populated on a window associated with the specific stage of the process. As such, the user may view, track, update, edit and/or save the ascertained information to the electronic database 220. In at least one other embodiment, the user may physically print an individual tracking sheet(s) 60 and/or an archive sheet(s) 70. As such, a user may print a complete or partially complete tracking sheet(s) 60 and may place it on a sheet retaining assembly 40 associated with an individual step of a process.

Reference to an element by the indefinite article “a” or “an” does not exclude the possibility that more than one element is present, unless the context clearly requires that there be one and only one element. The indefinite article “a” or “an” thus usually means “at least one.”

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

Thus, the examples set forth above are provided to give those of ordinary skill in the art a complete disclosure and description of how to make and use the embodiments of the disclosure and are not intended to limit the scope of what the inventors regard as the scope of the disclosure. Modifications of the above-described modes for carrying out the disclosure can be used by persons of skill in the art and are intended to be within the scope of the following claims.

It is to be understood that the disclosure is not limited to particular embodiments, which can, of course, vary. It is also to be understood that the terminology used herein is for the purpose of describing particular embodiments only and is not intended to be limiting.

A number of embodiments of the disclosure have been described. Nevertheless, it will be understood that various modifications may be made without departing from the spirit and scope of the present disclosure. Accordingly, other embodiments are within the scope of the following claims.

What is claimed is:

1. A system for tracking stages of a process, the system comprising:
a mounting assembly comprising:
a mounting bracket;
at least one connection structure disposed on a first side of the mounting bracket;
at least one attachment structure disposed on an interior side of the mounting bracket, the attachment operable to attach at least the mounting bracket to an intended surface;
a sheet retaining assembly comprising:
a support plate with a retainer operatively disposed thereon;
at least one connection structure disposed on a reverse side of the support plate;
the at least one connection structure of the mounting assembly operable with the at least one connection structure of the sheet retaining assembly to collectively define a removable engagement between them; and
a tracking assembly comprising an individual tracking sheet, the individual tracking sheet being removably disposable onto a front side of the support plate;
wherein the individual tracking sheet comprises a plurality of fields to monitor or update information associated with a fermented product during its production at one or more of the following stages:
a first stage comprising information relating to quantities to be added of one or more of a sulfite, an enzyme preparation, and a yeast nutrient;
a second stage comprising information relating to methods of application of a yeast;
a third stage comprising information relating to rehydration or the addition of fermentation nutrients;
a fourth stage comprising information relating to pressing the fermented product; and
a fifth stage comprising information relating to transferring the fermented product from a fermentation container to another container.

2. The system as recited in claim 1, wherein the tracking assembly further comprises an archive sheet for placement of the individual tracking sheet.

3. The system as recited in claim 1, wherein the mounting bracket comprises a plurality of segments collectively defining a substantially concave configuration of the mounting bracket.

4. The system as recited in claim 2, wherein the mounting bracket comprises a first segment adjacently disposed to a second segment, and a third segment adjacently disposed to the second segment.

5. The system as recited in claim 4, wherein the first segment, the second segment and the third segment collectively define cross section of the mounting bracket that is configured to substantially correspond to a curved geometry of the intended surface.

6. The system as recited in claim 1, wherein the at least one attachment structure is disposed on the interior side of the mounting bracket, and wherein the at least one attachment structure is disposable onto the intended surface.

7. The system as recited in claim 1, wherein the at least one connection structure of the mounting bracket is disposed on the exterior side of the mounting bracket.

8. The system as recited in claim 1, wherein the archive sheet comprises information associated with the resulting fermented product according to the process.

9. The system as recited in claim 1, wherein the fermented product is wine.

10. The system as recited in claim 1, wherein each of the at least one connecting structure of the mounting assembly and the at least one connecting structure of the sheet retaining assembly comprise magnets operable to define an engagement between them.

11. The system as recited in claim 1, further comprising at least a second mounting assembly with a substantially equivalent structure and attached to a different and corresponding intended surface; the sheet retaining assembly removably disposed into and out of the mounting assembly and the at least second mounting assembly.

12. A method of tracking stages of a process, the method comprising:
providing a system comprising at least an archive sheet and an individual tracking sheet with a plurality of fields to monitor or update information associated with a product produced according to the process;
placing the individual tracking sheet on an intended location associated with a first stage of the process;
updating the individual tracking sheet with product information associated with the first stage of the process;
moving the individual tracking sheet and placing it on another intended location associated with a subsequent stage of the process;
updating the individual tracking sheet with product information associated with the subsequent step of the process, and
placing the individual tracking sheet on the archive sheet.

13. The method as recited in claim 12, wherein the system comprises:
a mounting assembly comprising:
a mounting bracket;
at least one connection structure disposed on an exterior side of the mounting bracket;
at least one attachment structure disposed on an interior side of the mounting bracket, the attachment structure being operable to attach the mounting bracket to an intended surface;
a sheet retaining assembly comprising:
a support plate with a retainer operatively disposed thereon;
at least one connection structure disposed on a reverse side of the support plate;
the at least one connection structure of the mounting assembly operable with the at least one connection structure of the sheet retaining assembly to collectively define a removable engagement between them; and
a tracking assembly comprising the individual tracking sheet and the archive sheet, the individual tracking sheet removably disposable onto a front side of the support plate.

14. A system for tracking stages of a process, the system comprising:
a network operatively structured with a database and a server to store information associated with a product produced according to the process;
an electronic platform in communication implementable via a mobile device in communication with the database and the server via the network; the electronic platform being operable to allow a user to input or edit the information associated with the product;
wherein the electronic platform is operable with the mobile device to detect a digital component at an intended location of the process; and
wherein the digital component is associated with information about the product, or the intended location or stage of the process.

15. The system as recited in claim 14, wherein the electronic platform is operable with a camera of the mobile device to scan the digital component and retrieve information stored on the database or server relating to the intended location or stage of the process.

16. The system as recited in claim 15, wherein the electronic platform is operable to allow the user to input additional information or edit the retrieved information associated with the intended location or stage of the process.

17. The system as recited in claim 15, wherein the electronic platform is operable to automatically display an individual window on a screen of the mobile device presenting retrieved information relating to the intended location or stage of the process.

18. The system as recited in claim 14, wherein the system further comprises a tracking assembly comprising individual tracking sheet; and wherein the digital component is disposed onto the individual tracking sheet.

19. The system as recited in claim 18, wherein the individual tracking sheet is disposable onto a sheet retaining assembly disposable, and wherein the sheet retaining assembly is disposable onto an intended surface associated with the intended location or stage of the process.

20. The system as recited in claim 14, wherein the digital component is disposable directly onto an element associated with the intended location or stage of the process.
